(19) United States
(12) Patent Application Publication
(10) Pub. No.: US 2004/0181241 A1
(43) Pub. Date: Sep. 16, 2004

(54) Disposable sand-cap for a skin scrubbing apparatus

(76) Inventors: Jeom-Sup Jo, Jackson Heights, NY (US); Chang-Sung Ahn, Seoul (KR)

Correspondence Address: John S. Egbert, Harrison & Egbert, 7th Floor, 412 Main Street, Houston, TX, 77002, US

(21) Appl. No.: 10/418,614

(22) Filed: Apr. 21, 2003

(30) Foreign Application Priority Data
Mar. 12, 2003 (KR) 2003-0007333

(51) Int. Cl. A61B 17/50

(52) U.S. Cl. 606/131

(57) ABSTRACT

Disclosed is a disposable sand-cap for skin scrubbing apparatuses. The disposable sand-cap is assembled with an assembling body of a skin scrubbing apparatus, and includes a cap body including assembling grooves located at an upper part thereof and engaged with lugs, and grind grooves longitudinally and slantingly formed on an outer surface thereof. At this time, the cap body is made of a synthetic resin. A coarse grinding sand layer with sand-particles of 50 to 60 mesh is attached to the upper part of the outer surface of the cap body using an adhesive. Furthermore, a fine grinding sand layer with sand-particles of 90 to 100 mesh is attached to a lower part of the outer surface of the cap body using the adhesive. Accordingly, the disposable sand-cap is sufficiently competitive in terms of production cost because it consists of the synthetic resin, includes the coarse sand layer as well as the fine sand layer attached to the outer surface thereof to be desirably applied to various types of horniness, and is easily disassembled from a skin scrubbing apparatus and discarded after it is used one time, thereby securing advantages of not transmitting infection, in view of sanitation, and easiness of its use.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The present device pertains to a disposable sand-cap for skin scrubbing apparatuses. More particularly, the present device relates to a disposable sand-cap for skin scrubbing apparatuses, which is sufficiently competitive in terms of production cost because it consists of a synthetic resin, includes a coarse sand layer as well as a fine sand layer attached to an outer surface thereof to be desirably applied to various types of hominess, and is easily disassembled from a skin scrubbing apparatus and discarded after it is used one time, thereby securing advantages of not transmitting infection, in view of sanitation, and easiness of its use.

### BACKGROUND OF THE INVENTION

As well known to those skilled in the art, keratosis palmaris et plantaris is a kind of skin disease with which hominess is excessively formed on skins of palms and soles, and the cause of the skin disease depends on shapes and distributions of the horniness, and on inheritance patterns, attack time, concurrent abnormal symptoms, progress, and prognosis of the skin disease.

The hominess, that is, a hardened skin patch formed on the palms and soles due to the comification of epiderms of the palms and soles, is prone to be cracked during a cold season to injure a corium, so there is a need to eliminate the hominess before the corium is injured.

A conventional skin scrubbing apparatus used to eliminate the hominess comprises a grinding stone for eliminating the hominess (a portion corresponding to an assembling body 100 in accordance with the present device of FIG. 1) integrated with a shaft of a motor.

However, the conventional skin scrubbing apparatus is disadvantageous in that it is difficult to sufficiently eliminate skin remnants stuck to a surface of the grinding stone and they are very apt to decay even though the grinding stone is sterilized, so plural users are easily infected with various germs due to the decomposed skin remnants when the users use one conventional skin scrubbing apparatus in common.

Accordingly, use of the conventional skin scrubbing apparatus is prohibited so as to prevent infection at public parlors such as public baths and beauty shops in the United States of America and in some countries of Europe.

### BRIEF SUMMARY OF THE INVENTION

Therefore, the present device has been made keeping in mind the above disadvantages occurring in the prior arts, and an object of the present device is to provide a disposable sand-cap for skin scrubbing apparatuses, which is sufficiently competitive in terms of production cost because a cap body constituting the disposable sand-cap consists of a synthetic resin, includes a coarse sand layer as well as a fine sand layer attached to an outer surface of the cap body to be desirably applied to various types of horniness, and is easily disassembled from a skin scrubbing apparatus discarded after it is used one time, thereby securing advantages of no transmission of germs, in view of sanitation, and easiness of its use.

Based on the present device, the above object can be accomplished by providing a disposable sand-cap for skin scrubbing apparatuses including an assembling body assembled with a grinding sand layer, comprising a plurality of lugs outwardly protruded from the assembling body, and a cap body including assembling grooves located at an upper part thereof and engaged with the lugs, and grind grooves longitudinally and slantingly formed on an outer surface thereof. At this time, the cap body is made of a synthetic resin. A coarse grinding sand layer with sand-particles of 50 to 60 mesh is attached to the upper part of the outer surface of the cap body using an adhesive. Furthermore, a fine grinding sand layer with sand-particles of 90 to 100 mesh is attached to a lower part of the outer surface of the cap body using the adhesive.

The disposable sand-cap also includes a ring-shaped grinding band outwardly protruded from the lower part of outer surface of the cap body and having a diameter smaller than the upper part of the cap body.

### BRIEF DESCRIPTION OF THE SEVERAL VIEWS OF THE DRAWINGS

The above and other objects, features and other advantages of the present device will be more clearly understood from the following detailed description taken in conjunction with the accompanying drawings, in which:

FIG. 1 schematically illustrates a disposable sand-cap assembled with a skin scrubbing apparatus according to a first embodiment of the present device.

FIG. 2 schematically illustrates the engagement of an assembling body of the skin scrubbing apparatus into the disposable sand-cap of FIG. 1.

FIG. 3 is an enlarged sectional side view of the disposable sand-cap of FIG. 1 engaged with the assembling body of the skin scrubbing apparatus.

FIG. 4 is an enlarged sectional plan view of the assembling body of the skin scrubbing apparatus engaged with the disposable sand-cap of FIG. 1.

FIG. 5 is an enlarged sectional plan view of an assembling body of a skin scrubbing apparatus engaged with a disposable sand-cap according to a second embodiment of the present device; and

FIG. 6 schematically illustrates a manual skin scrubbing apparatus provided with the disposable sand-cap according to the present device.

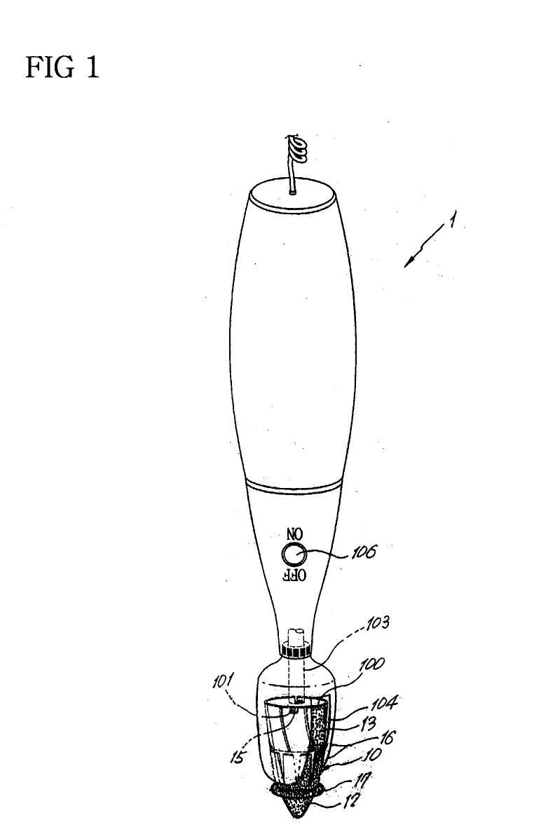

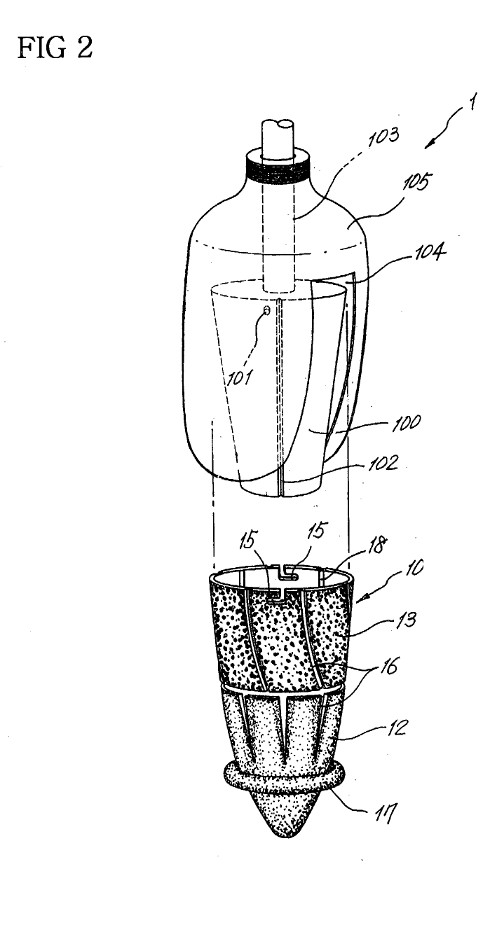

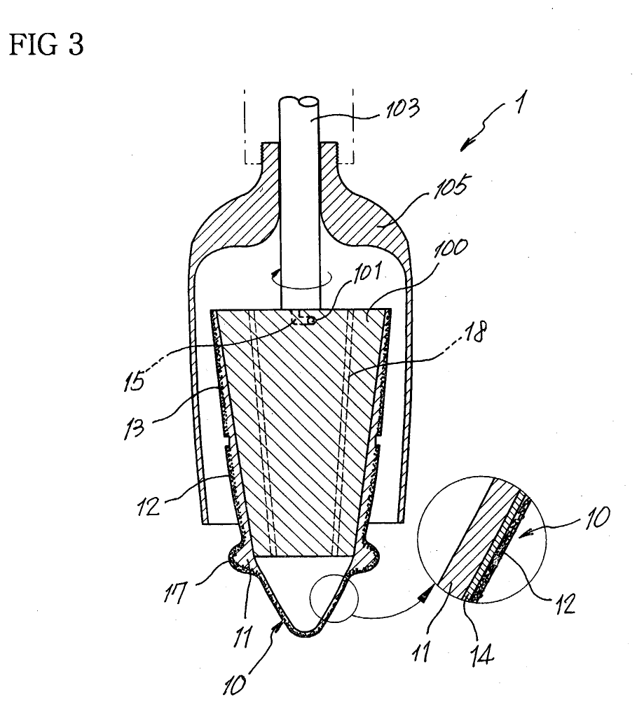

### DETAILED DESCRIPTION OF THE INVENTION

Reference now should be made to the drawings, in which the same reference numerals are used throughout the different drawings to designate the same or similar components.

A disposable sand-cap for a skin scrubbing apparatus according to a first embodiment of the present device is assembled with an automatic skin scrubbing apparatus 1 or a manual skin scrubbing apparatus 2. In the case of the automatic skin scrubbing apparatus 1, the disposable sand-cap 10 rotates by a motor to eliminate hominess, but, in the manual skin scrubbing apparatus 2, the disposable sand-cap 10 assembled with a handle 200 is manually moved to eliminate the hominess.

As for the automatic skin scrubbing apparatus 1, the disposable sand-cap 1 is engaged with an assembling body 100 of the apparatus 1 with the use of lugs and rotates in conjunction with the assembling body 100 by a motor (not shown), and comprises a cap body 11 attached with a grinding sand layer consisting of emery using an adhesive 14, a plurality of grind grooves 16 longitudinally and slantingly formed on an outer surface of the cap body 11 to increase abrasiveness of the cap body 11 against the hominess and receive skin remnants separated from hominess, a grinding band 17 outwardly protruded from a lower part of the cap body 11, and a plurality of combination prominences 18 inwardly protruded from an inner surface of the sand-cap 10. At this time, the skin remnants in the grind grooves 16 no longer accumulate in the sand-cap 10 when they are accumulated more than a predetermined amount within the grind grooves 16.

The lugs 101 protruded from diametrically opposite sides around an upper part of the assembling body 100 are inserted into assembling grooves 15 formed at an upper part of the sand-cap 10 and rotate slightly to allow the assembling body 100 to be engaged with the sand-cap 10.

The sand-cap 10 is used only one time and then discarded, and consists of a hard synthetic resin such as hard PVC (polyvinyl chloride) Additionally, the sand-cap 10 comprises a coarse grinding sand layer 13 with sand-particles of 50 to 60 mesh attached to the upper part of cap body 11 by the adhesive 14, and a fine grinding sand layer 12 with sand-particles of 90 to 100 mesh attached to a lower part of the cap body 11 by the adhesive 14. Alternatively, a middle-sized grinding sand layer may be attached to a whole outer surface of the cap body 11 with the use of the adhesive 14.

Useful as the adhesive 14 in the present device is a low-temperature adhesive.

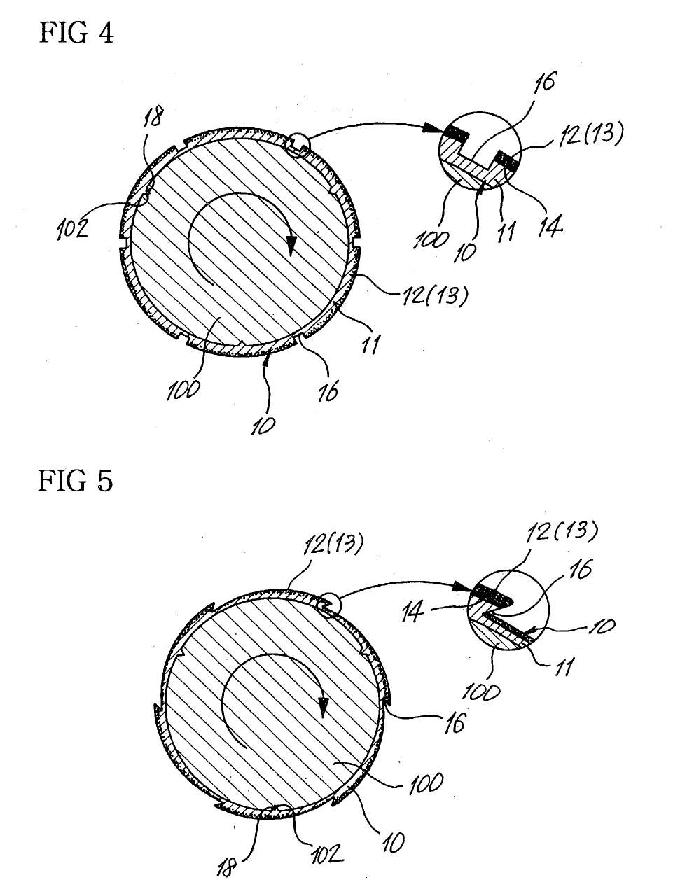

As described above, the grind grooves 16 longitudinally and slantingly formed on the outer surface of the cap body 11 function to increase abrasiveness of the cap body 11 against the hominess, and act as channels for receiving the ground skin remnants and discharging the accumulated skin remnants therethrough. As in FIG. 4, the grind grooves 16 are formed on the outer surface of the body 11 in such a way that walls of the grind grooves 16 are at an angle of 90 degrees with bottoms of the grind grooves 16 to increase the abrasiveness of the outer surface of the cap body 11 against the hominess during rotation of the sand-cap 10.

Combination grooves 102 are longitudinally formed on an outer surface of the assembling body 100, and combination prominences 18 corresponding to the combination grooves 102 are formed on an inner surface of the sand-cap 10. Accordingly, the assembling body 100 is inserted into the sand-cap 10 in such a way that the combination prominences 18 are engaged with the combination grooves 102 of the assembling body 100 to allow the sand-cap 10, consisting of the hard synthetic resin such as hard PVC and surrounding the assembling body 100, to smoothly rotate in conjunction with the assembling body 100 without distortion of the sand-cap 10.

With reference to FIG. 5, there is illustrated an enlarged sectional plan view of an assembling body of a skin scrubbing apparatus engaged with a disposable sand-cap according to a second embodiment of the present device, in which a first wall of each of grind grooves 16 is inclined at an acute angle with a bottom of each of the grind grooves 11 and a second wall of each of the grind grooves 16 is gradually extended to an outer surface of a cap body 11, thus the outer surface of the cap body 11 forms a sawtooth shape. Thereby, the grind grooves 16 contribute to effectively eliminating hominess during rotation of the cap body 11 due to increase abrasiveness of the cap body 11 against the horniness.

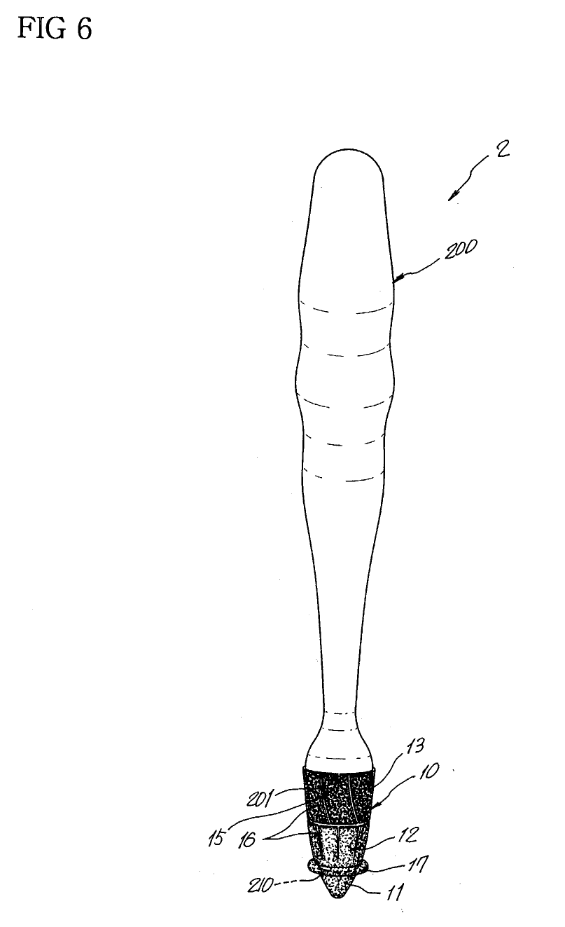

Referring to FIG. 6, there is illustrated a manual skin scrubbing apparatus provided with the disposable sand-cap according to the present device. The manual skin scrubbing apparatus 2 comprises a handle 200 having a lower part with a trapezoidal side sectional shape, and an assembling body 210 having lugs 201 outwardly protruded from any one side or diametrically opposite sides around the outer surface thereof. At this time, a surface of the handle 200 is uneven to allow users to firmly grasp the handle 200, so it is convenient to handle the sand-cap 10 assembled with the assembling body 210 by moving the handle 200.

Now, there will be described the operation of the disposable sand-cap for the skin scrubbing apparatuses according to the present device.

Firstly, the disposable sand-cap 1 is produced. A method of producing the sand-cap 1 comprises the steps of forming the cap body 11 consisting of the hard synthetic resin such as hard PVC, and attaching the grinding sand layer such as emery to the whole surface of the cap body 11 except grind grooves 16 using the adhesive 14 in such a way that the coarse grinding sand layer 13 with sand-particles of 50 to 60 mesh is attached to the upper part of the cap body 11 and the fine grinding sand layer 12 with sandparticles of 90 to 100 mesh is attached to the lower part of the cap body 11.

After the disposable sand-cap 10 thus produced is positioned to vertically correspond to the lower part of the assembling body 100 assembled with an end of the shaft 103 of the motor of the automatic skin scrubbing apparatus 1, the lower part of the assembling body 100 is inserted into the sand-cap 10 while the lugs 101 of the assembling body 100 are engaged with the assembling grooves 15 of the upper part of the sand-cap 10, and the sand-cap 10 is slightly rotated while the assembling body 100 is embedded in the sand-cap 10 so as to firmly engage the lugs 101 of the assembling body 100 with the assembling grooves 15 of the sand-cap 10.

Furthermore, the combination prominences 18 on the inner surface of the sand-cap 10 are engaged with the combination grooves 102 of the assembling body 100, thereby preventing the assembling body 100 from running idle in the sand-cap 10.

After the completion of assembling the assembling body 100 with the sand-cap 10, a user pushes an on-off switch 106 of the automatic skin scrubbing apparatus 1 to rotate the assembling body 100 integrated with the sand-cap 10 in a clockwise direction in accordance with rotation of the shaft 103 of the motor.

The user then applies the sand-cap 10 to the hominess on the palms or soles to eliminate it from the palms or soles.

After grinding a hardened skin using the coarse grinding sand layer 13 of the sand-cap 10, the user may additionally and carefully grind the ground skin using the fine grinding sand layer 12.

Skin pieces ground by the sand-cap 10 are accumulated in the grind grooves 16 formed on the outer surface of the sand-cap 10, and the hominess pieces are discharged outside the sand-cap 105 or into a grind body 105 when accumulated more than a predetermined amount.

At this time, the hominess is eliminated from only a skin region exposed to a grind window 104 of the grind body 105 of the automatic skin scrubbing apparatus 1, so most of the skin remnants collect inside the grind body 105 and safety in using the automatic skin scrubbing apparatus 1 is secured.

Moreover, the sand sand-cap 10 is slightly pointed at its terminal end, so it is convenient to eliminate a small area of horniness.

Particularly, the grinding band 17 outwardly protruded from a lower part of the fine grinding sand layer 12 is usefully used to eliminate a long, thin region of hardened skin.

Further, the sawtooth-shaped sand-cap 10 is effectively used to eliminate the coarse and thick hardened skin as shown in FIG. 5.

After using the disposable sand-cap 10, the sand-cap is rotated around the assembling body 100 in a counterclockwise direction to separate the lugs 101 of the assembling body 100 from the assembling grooves 15 of the sand-cap 10, and then pulled out from the assembling body 100 to be disassembled from the assembling body 100. The used sand-cap 10 is discarded.

As for the manual skin scrubbing apparatus 2 as shown in FIG. 6, the disposable sand-cap 10 is sand-capped on the assembling body 210 assembled with the handle 200 of the manual skin scrubbing apparatus 2 in such a way that the lugs 201 of the assembling body 210 of the handle 200 are engaged with the assembling grooves 15 of the sand-cap 10 and the sand-cap 10 is then slightly rotated so as to firmly engage the lugs 201 with the assembling grooves 15. Therefore, the user can eliminate the hominess by rubbing the sand-cap 10 assembled with the handle 200 against the hardened skin of the palms or soles.

As described above, the present device provides a disposable sand-cap for skin scrubbing apparatuses, which is sufficiently competitive in terms of production cost because it consists of a synthetic resin, includes a coarse sand layer as well as a fine sand layer attached to an outer surface thereof to be desirably applied to various types of horniness, and is easily disassembled from a skin scrubbing apparatus and discarded after it is used one time, thereby securing advantages of no transmission of germs, in view of sanitation, and easiness of its use.

The present device has been described in an illustrative manner, and it is to be understood that the terminology used is intended to be in the nature of description rather than of limitation. Many modifications and variations of the present device are possible in light of the above teachings. Therefore, it is to be understood that within the scope of the appended claims, the device may be practiced otherwise than as specifically described.

We claim:

1. A disposable sand-cap for skin scrubbing apparatuses including an assembling body assembled with a grinding sand layer, comprising:
a plurality of lugs outwardly protruded from the assembling body;
a cap body including assembling grooves located at an upper part thereof and engaged with the lugs, and grind grooves longitudinally and slantingly formed on an outer surface thereof, said cap body being made of a synthetic resin;
a coarse grinding sand layer with sand-particles of 50 to 60 mesh attached to the upper part of the outer surface of the cap body using an adhesive; and
a fine grinding sand layer with sand-particles of 90 to 100 mesh attached to the lower part of the outer surface of the cap body using an adhesive.

2. The disposable sand-cap as set forth in claim 1, further comprising a ring-shaped grinding band outwardly protruded from the lower part of the outer surface of the cap body and having a diameter smaller than the upper part f the cap body.
